(19) United States
(12) Patent Application Publication
(10) Pub. No.: US 2012/0082731 A1
(43) Pub. Date: Apr. 5, 2012

(54) Method For Removing Residual Organic Solvent From Microparticles

(76) Inventors: Adrian Raiche, Helena, AL (US); Brenda Perkins, Brimingham, AL (US)

(21) Appl. No.: 13/247,509

(22) Filed: Sep. 28, 2011

Related U.S. Application Data
(60) Provisional application No. 61/388,050, filed on Sep. 30, 2010.
(60) Provisional application No. 61/388,049, filed on Sep. 30, 2010.

(51) Int. Cl. A61K 9/50; A61K 38/08; F26B 3/00; A61K 38/00

(52) U.S. Cl. 424/499; 514/1.1; 514/21.7; 34/282

(57) ABSTRACT

Disclosed herein are methods comprising suspending microparticles in a surfactant/non-polar alkane solution to remove residual solvent that is present in the microparticle.

This application claims benefit of U.S. Provisional Application No. 61/388,050, filed Sep. 30, 2011, which is hereby incorporated herein by reference in its entirety.

### BACKGROUND

Microparticles are often prepared using a solvent to dissolve the polymer which forms the microparticle matrix. Typical solvents for polyesters such as lactide and/or glycolide based polymers include a variety of ICH Class I and Class II solvents, such as chlorinated solvents. Such solvents are regulated and cannot be present above certain amounts in formulations for in vivo use. In many microparticle production processes, however, residual solvent is difficult to remove. Accordingly, a need exists for methods to overcome the problem of residual solvent.

### SUMMARY

Disclosed herein are methods of overcoming the problem of residual solvent comprising: (a) mixing microparticles with a surfactant in a non-polar alkane to provide a dispersion; wherein the microparticles comprise at least 2% by weight residual organic solvent; (b) stirring the dispersion; (c) collecting the microparticles; (d) rinsing the microparticles; and (e) drying the microparticles.

### DETAILED DESCRIPTION

In this specification and in the claims that follow, reference will be made to a number of terms that shall be defined to have the following meanings:

Throughout this specification, unless the context requires otherwise, the word “comprise,” or variations such as “comprises” or “comprising,” will be understood to imply the inclusion of a stated integer or step or group of integers or steps but not the exclusion of any other integer or step or group of integers or steps.

The singular forms “a,” “an” and “the” include plural referents unless the context clearly dictates otherwise. Thus, for example, reference to “a bioactive agent” includes mixtures of two or more such agents, and the like.

“Biodegradable” refers to materials that erode to soluble species or that degrade under physiologic conditions to smaller units or chemical species that are, themselves, non-toxic (biocompatible) to the subject and capable of being metabolized, eliminated, or excreted by the subject.

The term “microparticle” is used herein to refer generally to a variety of structures having sizes from about 10 nm to 2000 microns (2 millimeters) and includes microcapsule, microsphere, nanoparticle, nanocapsule, nanosphere as well as particles, in general, that are less than about 2000 microns (2 millimeters).

A “bioactive agent” refers to an agent that has biological activity. The biological agent can be used to treat, diagnose, cure, mitigate, prevent (i.e., prophylactically), ameliorate, modulate, or have an otherwise favorable effect on a disease, disorder, infection, and the like. Bioactive agents also include those substances which affect the structure or function of a subject, or a pro-drug, which becomes bioactive or more bioactive after it has been placed in a predetermined physiological environment.

In one aspect, disclosed herein are methods of overcoming the problem of residual solvent comprising: (a) mixing microparticles with a surfactant in a non-polar alkane to provide a dispersion; wherein the microparticles comprise at least 2% by weight residual organic solvent; (b) stirring the dispersion; (c) collecting the microparticles; (d) rinsing the microparticles; and (e) drying the microparticles.

The methods disclosed herein involve suspending microparticles in a non-polar alkane surfactant solution to remove residual organic solvent from the microparticles. The organic solvent that is present in the microparticles can be residual from a microparticle preparation process, such as an emulsion process, wherein an organic solvent can be used as a solvent for the polymer and/or the bioactive agent or other excipient. The non-polar alkane can be miscible with the residual organic solvent, but does not solubilize polymers of the microparticle. However, the non-polar alkane acts as a plasticizer and soaks into the microparticles, which allows for residual organic solvent, such as a chlorinated solvent or ethyl acetate, to diffuse into the heptane solution. The microparticles are then washed with a surfactant-free non-polar alkane, such as surfactant-free heptane. The microparticles can also be rinsed with water, or water and a non-polar alkane, such as heptane (including surfactant-free heptane), and dried.

The residual organic solvent can comprise a C1-C4 halogenated alkane (such as, for example, a C1-C4 chlorinated alkane), ethyl acetate, or a combination thereof. The residual organic solvent can be any suitable solvent, including without limitation methylene chloride, chloroform, carbon tetrachloride, ethylene dichloride, ethylene chloride, 2,2,2-trichloroethane, or a mixture thereof.

The non-polar alkane solution has a plasticizing effect on the microparticles particularly when the microparticles comprise lactide and/or glycolide. When lactide or glycolide based microparticles are suspended in water (after preparing the microparticles), the stressed organization of polymers creates a large microparticle pore structure and increases diffusivity of substances out of and into the microparticles. This, in turn, leads to an initial burst of an encapsulated substance. The non-polar alkane solution, however, relaxes the internal stress of the polymers and therefore reduces water ingress and initial burst. The non-polar alkane, such as heptane, can be used to increase or decrease release of an agent in the microparticles (modulate release) while also decreasing residual organic solvent levels. In some examples, the bioactive agent has slight or partial solubility in the non-polar alkane, for example, at least 0.1 mg/mL, or from 0.1 mg/mL to 0.5 mg/mL. In such cases, the burst or release of the bioactive agent from the microparticle can be accelerated.

In contrast to existing phase separation techniques, the disclosed method does not involve the use of oils such as silicon oils. Silicone oils are often used in microparticle phase separation processes. However, silicone oil can be difficult to entirely remove, contaminates surfaces, and can be difficult to discard. The disclosed method also allows for the ability to exchange an ICH Class II solvent, such as dichloromethane or ethyl acetate, with a class III solvent, such as heptane. Residual non-polar alkane (such as heptane) that may be present in the microparticles after carrying out the method is not as great of a concern as residual dichloromethane or ethyl acetate.

As briefly discussed above, the disclosed methods comprise first mixing microparticles with a surfactant solution in a non-polar alkane to provide a dispersion of microparticles in the non-polar alkane solution. The surfactant can be added to the non-polar alkane prior to the addition of the microparticles. The surfactant functions to disperse the microparticles such that the non-polar alkane can effectively soak and/or penetrate the microparticle matrix.

A variety of surfactants can be used. Examples of surfactants include sorbitol monostearate (also known as SPAN), sorbitan monostearate (also known as SPAN 60), sorbitan monooleate (SPAN 80), polyoxyethylene sorbitan monooleate (TWEEN 80), all of which are commercially available. It is understood and herein contemplated that the surfactant mixture can comprise any one surfactant or combination of two, three, four or more surfactants. For example, the surfactant mixture can comprise sorbitol monostearate and sorbitan monostearate, sorbitol monostearate and sorbitan monooleate; sorbitol monostearate and polyoxyethylene sorbitan monooleate, sorbitan monostearate and sorbitan monooleate, sorbitan monostearate and polyoxyethylene sorbitan monooleate, sorbitan monooleate and polyoxyethylene sorbitan monooleate, sorbitol monostearate, sorbitan monostearate, and sorbitan monooleate, sorbitol monostearate, sorbitan monostearate, and polyoxyethylene sorbitan monooleate, or any other combination of the above identified surfactants. The non-polar alkane solution can comprise at least 0.5% surfactant, for example from 0.5% to 10%, 0.5% to 8%, 0.5% to 6%, 0.5% to 5%, or 0.5% to 2%.

The non-polar alkane can be a variety of alkanes having from 1 to 24 carbons. The alkanes can be branched or unbranched, cyclic, or non-cyclic. Examples include, without limitation, pentane, cyclopentane, hexanes, cyclohexane, and heptane. “Hexanes” refers to commercially available hexanes, which includes a variety of isomers of hexane (all having the formula, C₆H₁₄), and is thus referred to as “hexanes,” rather than “hexane.”

Prior to being added the non-polar alkane solution, the microparticles comprise some amount of residual solvent left over from a production process, such as an emulsion process. In one aspect, the disclosed methods can be useful for microparticles that comprise at least 2% by weight residual organic solvent, for example from 2% to 5%. The residual organic solvent can be the solvent used as the solvent for the polymer during the microparticle production process. The non-polar alkane, in contrast, is not a solvent for the polymer from which the microparticles were formed. The non-polar alkane is also not a solvent for any bioactive agent or excipient present in the microparticles. After carrying out the disclosed methods, residual solvent amounts can be reduced.

After being adding the non-polar alkane solution, the dispersion of microparticles can be stirred for a period of time generally ranging from a 5 minutes to 4 hours, for example, from 30 minutes to 2 hours. After stirring the dispersion, the microparticles can be collected, for example by filtration or by sieve separation. Once the microparticles are collected, the microparticles can be rinsed with surfactant-free non-polar alkane, such as heptane, water, or a combination thereof, and dried. The drying step can be carried out using methods known in the art, such as spray-drying, air-drying, vacuum filtration, and the like.

A variety of microparticle production processes utilize one or more solvents for the polymer from which the microparticles are formed. These methods generally include, without limitation, film casting, molding, spray drying and extrusion, or emulsion or double-emulsion processes. In one aspect, the disclosed methods can be particularly useful for microparticles prepared using emulsion or double-emulsion techniques. Emulsion methods for preparing microparticles are discussed in Jeffery, et al., “The preparation and characterisation of poly(lactide-co-glycolide) microparticles. I: Oil-In-water emulsion solvent evaporation,” Int. J. Pharm. 77(2-3):169-175 (1991); Jeffery, et al., “The Preparation and Characterization of Poly(lactide-co-glycolide) Microparticles. II. The Entrapment of a Model Protein using a (Water-in-Oil)-in-Water Emulsion Solvent Evaporation Technique,” Pharm. Res. 10(3):362-368 (1993). Solvent evaporation methods are discussed Wichert, B. and Rohdewald, P. (1993) J. Microencapsul. 10:195. Solvent extraction methods are described in U.S. Pat. No. 5,407,609, the entirety of which is incorporated herein by reference.

The microparticles can comprise a variety of biocompatible or biodegradable polymers. The biocompatible polymer can also be a biodegradable polymer. In one aspect, the biocompatible polymer can be one or more of polyesters, polyhydroxyalkanoates, polyhydroxybutyrates, polydioxanones, polyhydroxyvalerates, polyanhydrides, polyorthoesters, polyphosphazenes, polyphosphates, polyphosphoesters, polydioxanones, polyphosphoesters, polyphosphates, polyphosphonates, polyphosphates, polyhydroxyalkanoates, polycarbonates, polyalkylcarbonates, polyorthocarbonates, polyesteramides, polyamides, polyamines, polypeptides, polyurethanes, polyalkylene alkylates, polyalkylene oxalates, polyalkylene succinates, polyhydroxy fatty acids, polyacetals, polycyanoacrylates, polyketals, polyetheresters, polyethers, polyalkylene glycols, polyalkylene oxides, polyethylene glycols, polyethylene oxides, polypeptides, polysaccharides, or polyvinyl pyrrolidones. Other non-biodegradable but durable and bioacompatible polymers include without limitation ethylene-vinyl acetate co-polymer, polytetrafluoroethylene, polypropylene, polyethylene, and the like. Likewise, other suitable non-biodegradable polymers include without limitation silicones and polyurethanes.

The biocompatible and/or biodegradable polymer can be a poly(lactide), a poly(glycolide), a poly(lactide-co-glycolide), a poly(caprolactone), a poly(orthoester), a poly(phosphazene), a poly(hydroxybutyrate) or a copolymer containing a poly(hydroxybutarate), a poly(lactide-co-caprolactone), a polycarbonate, a polyesteramide, a polyanhydride, a poly(dioxanone), a poly(alkylene alkylate), a copolymer of polyethylene glycol and a polyorthoester, a biodegradable polyurethane, a poly(amino acid), a polyamide, a polyesteramide, a polyetherester, a polyacetal, a polycyanoacrylate, a poly(oxyethylene)/poly(oxypropylene) copolymer, polyacetals, polyketals, polyphosphoesters, polyhydroxyvalerates or a copolymer containing a polyhydroxyvalerate, polyalkylene oxalates, polyalkylene succinates, poly(maleic acid), and copolymers, terpolymers, combinations, or blends thereof.

The biocompatible or biodegradable polymer can comprise any lactide residue, including all racemic and stereospecific forms of lactide, including, but not limited to, L-lactide, D-lactide, and D,L-lactide, or a mixture thereof. Useful polymers comprising lactide include, but are not limited to poly(L-lactide), poly(D-lactide), and poly(DL-lactide); and poly(lactide-co-glycolide), including poly(L-lactide-co-glycolide), poly(D-lactide-co-glycolide), and poly(DL-lactide-co-glycolide); or copolymers, terpolymers, combinations, or blends thereof. Lactide/glycolide polymers can be conveniently made by melt polymerization through ring opening of lactide and glycolide monomers. Additionally, racemic DL-lactide, L-lactide, and D-lactide polymers are commercially available. The L-polymers are more crystalline and resorb slower than DL-polymers. In addition to copolymers comprising glycolide and DL-lactide or L-lactide, copolymers of L-lactide and DL-lactide are commercially available. Homopolymers of lactide or glycolide are also commercially available.

When the biodegradable and/or biocompatible polymer is poly(lactide-co-glycolide), poly(lactide), or poly(glycolide), the amount of lactide and glycolide in the polymer can vary. In a further aspect, the biodegradable polymer contains 0 to 100 mole %, 40 to 100 mole %, 50 to 100 mole %, 60 to 100 mole %, 70 to 100 mole %, or 80 to 100 mole % lactide and from 0 to 100 mole %, 0 to 60 mole %, 10 to 40 mole %, 20 to 40 mole %, or 30 to 40 mole % glycolide, wherein the amount of lactide and glycolide is 100 mole %. In a further aspect, the biodegradable polymer can be poly(lactide), 95:5 poly(lactide-co-glycolide) 85:15 poly(lactide-co-glycolide), 75:25 poly(lactide-co-glycolide), 65:35 poly(lactide-co-glycolide), or 50:50 poly(lactide-co-glycolide), where the ratios are mole ratios.

The biodegradable and/or biocompatible polymer can also be a poly(caprolactone) or a poly(lactide-co-caprolactone). The polymer can be a poly(lactide-caprolactone), which, in various aspects, can be 95:5 poly(lactide-co-caprolactone), 85:15 poly(lactide-co-caprolactone), 75:25 poly(lactide-co-caprolactone), 65:35 poly(lactide-co-caprolactone), or 50:50 poly(lactide-co-caprolactone), where the ratios are mole ratios.

A variety of bioactive agents or other excipients can be present in the microparticles. The bioactive agents disclosed herein can be water soluble or have at least partial solubility in the non-polar alkanes disclosed herein.

Examples of bioactive agents include without limitation small molecules, peptides, oligopeptides (e.g., octreotide), proteins such as hormones, enzymes, antibodies, receptor binding proteins, antibody fragments, antibody conjugates, nucleic acids such as aptamers, iRNA, siRNA, microRNA, DNA, RNA, antisense nucleic acid or the like, antisense nucleic acid analogs or the like, VEGF inhibitors, macrocyclic lactones, dopamine agonists, dopamine antagonists, low-molecular weight compounds, high-molecular-weight compounds, or conjugated bioactive agents.

Other bioactive agents can include anabolic agents, antacids, anti-asthmatic agents, anti-cholesterolemic and anti-lipid agents, anti-coagulants, anti-convulsants, anti-diarrheals, anti-emetics, anti-infective agents including antibacterial and antimicrobial agents, anti-inflammatory agents, anti-manic agents, antimetabolite agents, anti-nauseants, anti-neoplastic agents, anti-obesity agents, antipsychotics, anti-pyretic and analgesic agents, anti-spasmodic agents, anti-thrombotic agents, anti-tussive agents, anti-uricemic agents, anti-anginal agents, antihistamines, appetite suppressants, biologicals, cerebral dilators, coronary dilators, bronchiodilators, cytotoxic agents, decongestants, diuretics, diagnostic agents, erythropoietic agents, expectorants, gastrointestinal sedatives, hyperglycemic agents, hypnotics, hypoglycemic agents, immunomodulating agents, ion exchange resins, laxatives, mineral supplements, mucolytic agents, neuromuscular drugs, peripheral vasodilators, psychotropics, sedatives, stimulants, thyroid and anti-thyroid agents, tissue growth agents, uterine relaxants, vitamins, or antigenic materials.

Still other bioactive agents include androgen inhibitors, polysaccharides, growth factors, hormones, anti-angiogenesis factors, dextromethorphan, dextromethorphan hydrobromide, noscapine, carbetapentane citrate, chlophedianol hydrochloride, chlorpheniramine maleate, phenindamine tartrate, pyrilamine maleate, doxylamine succinate, phenyltoloxamine citrate, phenylephrine hydrochloride, phenylpropanolamine hydrochloride, pseudoephedrine hydrochloride, ephedrine, codeine phosphate, codeine sulfate morphine, mineral supplements, cholestryramine, N-acetylprocainamide, acetaminophen, aspirin, ibuprofen, phenyl propanolamine hydrochloride, caffeine, guaifenesin, aluminum hydroxide, magnesium hydroxide, peptides, polypeptides, proteins, amino acids, hormones, interferons, cytokines, and vaccines.

Representative drugs that can be used as bioactive agents include, but are not limited to, peptide drugs, protein drugs, therapeutic antibodies, anticalins, desensitizing materials, antigens, anti-infective agents such as antibiotics, antimicrobial agents, antiviral, antibacterial, antiparasitic, antifungal substances and combination thereof, antiallergenics, androgenic steroids, decongestants, hypnotics, steroidal anti-inflammatory agents, anti-cholinergics, sympathomimetics, sedatives, miotics, psychic energizers, tranquilizers, vaccines, estrogens, progestational agents, humoral agents, prostaglandins, analgesics, antispasmodics, antimalarials, antihistamines, cardioactive agents, anti-inflammatory agents, nonsteroidal anti-inflammatory agents, antiparkinsonian agents, antihypertensive agents, β-adrenergic blocking agents, nutritional agents, anti-TNF agents and the benzophenanthridine alkaloids. The agent can further be a substance capable of acting as a stimulant, sedative, hypnotic, analgesic, anticonvulsant, and the like.

Other bioactive agents include but are not limited to analgesics such as acetaminophen, acetylsalicylic acid, and the like; anesthetics such as lidocaine, xylocalne, and the like; anorexics such as dexadrine, phendimetrazine tartrate, and the like; antiarthritics such as methylprednisolone, ibuprofen, and the like; antiasthmatics such as terbutaline sulfate, theophylline, ephedrine, and the like; antibiotics such as sulfisoxazole, penicillin G, ampicillin, cephalosporins, amikacin, gentamicin, tetracyclines, chloramphenicol, erythromycin, clindamycin, isoniazid, rifampin, and the like; antifungals such as amphotericin B, nystatin, ketoconazole, and the like; antivirals such as acyclovir, amantadine, and the like; anticancer agents such as cyclophosphamide, methotrexate, etretinate, and the like; anticoagulants such as heparin, warfarin, and the like; anticonvulsants such as phenyloin sodium, diazepam, and the like; antidepressants such as isocarboxazid, amoxapine, and the like; antihistamines such as diphenhydramine HCl, chlorpheniramine maleate, and the like; antipsychotics such as clozapine, haloperidol, carbamazepine, gabapentin, topimarate, bupropion, sertraline, alprazolam, buspirone, risperidone, aripiprazole, olanzapine, quetiapine, ziprasidone, iloperidone, and the like; hormones such as insulin, progestins, estrogens, corticoids, glucocorticoids, androgens, and the like; tranquilizers such as thorazine, diazepam, chlorpromazine HCl, reserpine, chlordiazepoxide HCl, and the like; antispasmodics such as belladonna alkaloids, dicyclomine hydrochloride, and the like; vitamins and minerals such as essential amino acids, calcium, iron, potassium, zinc, vitamin B12, and the like; cardiovascular agents such as prazosin HCl, nitroglycerin, propranolol HCl, hydralazine HCl, pancrelipase, succinic acid dehydrogenase, and the like; peptides and proteins such as LHRH, somatostatin, calcitonin, growth hormone, glucagon-like peptides, growth releasing factor, angiotensin, FSH, EGF, bone morphogenic protein (BMP), erythopoeitin (EPO), interferon, interleukin, collagen, fibrinogen, insulin, Factor VIII, Factor IX, Enbrel®, Rituxan®, Herceptin®, alpha-glucosidase, Cerazyme/Ceredose®, vasopressin, ACTH, human serum albumin, gamma globulin, structural proteins, blood product proteins, complex proteins, enzymes, antibodies, monoclonal antibodies, and the like; prostaglandins; nucleic acids; carbohydrates; fats; narcotics such as morphine, codeine, and the like, psychotherapeutics; anti-malarials, L-dopa, diuretics such as furosemide, spironolactone, and the like; antiulcer drugs such as rantidine HCl, cimetidine HCl, and the like.

The bioactive agent can also be an immunomodulator, including, for example, cytokines, interleukins, interferon, colony stimulating factor, tumor necrosis factor, and the like; allergens such as cat dander, birch pollen, house dust mite, grass pollen, and the like; antigens of bacterial organisms such as Streptococcus pneumoniae, Haemophilus influenzae, Staphylococcus aureus, Streptococcus pyrogenes, Corynebacterium diphteriae, Listeria monocytogenes, Bacillus anthracis, Clostridium tetani, Clostridium botulinum, Clostridium perfringens. Neisseria meningitides, Neisseria gonorrhoeae, Streptococcus mutans. Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptspirosis interrogans, Borrelia burgddorferi, Campylobacter jejuni, and the like; antigens of such viruses as smallpox, influenza A and B, respiratory synctial, parainfluenza, measles, HIV, SARS, varicella-zoster, herpes simplex 1 and 2, cytomeglavirus, Epstein-Barr, rotavirus, rhinovirus, adenovirus, papillomavirus, poliovirus, mumps, rabies, rubella, coxsackieviruses, equine encephalitis, Japanese encephalitis, yellow fever, Rift Valley fever, lymphocytic choriomeningitis, hepatitis B, and the like; antigens of such fungal, protozoan, and parasitic organisms such as Cryptococcuc neoformans, Histoplasma capsulatum, Candida albicans, Candida tropicalis, Nocardia asteroids, Rickettsia ricketsii, Rickettsia typhi, Mycoplasma pneumoniae, Chlamyda psittaci, Chlamydia trachomatis, Plasmodium falciparum, Trypanasoma brucei, Entamoeba histolytica, Toxoplasma gondii, Trichomonas vaginalis, Schistosoma mansoni, and the like. These antigens can be in the form of whole killed organisms, peptides, proteins, glycoproteins, carbohydrates, or combinations thereof.

In a further specific aspect, the bioactive agent can comprise an antibiotic. The antibiotic can be, for example, one or more of Amikacin, Gentamicin, Kanamycin, Neomycin, Netilmicin, Streptomycin, Tobramycin, Paromomycin, Ansamycins, Geldanamycin, Herbimycin, Carbacephem, Loracarbef, Carbapenems, Ertapenem, Doripenem, Imipenem/Cilastatin, Meropenem, Cephalosporins (First generation), Cefadroxil, Cefazolin, Cefalotin or Cefalothin, Cefalexin, Cephalosporins (Second generation), Cefaclor, Cefamandole, Cefoxitin, Cefprozil, Cefuroxime, Cephalosporins (Third generation), Cefixime, Cefdinir, Cefditoren, Cefoperazone, Cefotaxime, Cefpodoxime, Ceftazidime, Ceftibuten, Ceftizoxime, Ceftriaxone, Cephalosporins (Fourth generation), Cefepime, Cephalosporins (Fifth generation), Ceftobiprole, Glycopeptides, Teicoplanin, Vancomycin, Macrolides, Azithromycin, Clarithromycin, Dirithromycin, Erythromycin, Roxithromycin, Troleandomycin, Telithromycin, Spectinomycin, Monobactams, Aztreonam, Penicillins, Amoxicillin, Ampicillin, Azlocillin, Carbenicillin, Cloxacillin, Dicloxacillin, Flucloxacillin, Mezlocillin, Meticillin, Nafcillin, Oxacillin, Penicillin, Piperacillin, Ticarcillin, Polypeptides, Bacitracin, Colistin, Polymyxin B, Quinolones, Ciprofloxacin, Enoxacin, Gatifloxacin, Levofloxacin, Lomefloxacin, Moxifloxacin, Norfloxacin, Ofloxacin, Trovafloxacin, Sulfonamides, Mafenide, Prontosil (archaic), Sulfacetamide, Sulfamethizole, Sulfanilimide (archaic), Sulfasalazine, Sulfisoxazole, Trimethoprim, Trimethoprim-Sulfamethoxazole (Co-trimoxazole) (TMP-SMX), Tetracyclines, including Demeclocycline, Doxycycline, Minocycline, Oxytetracycline, Tetracycline, and others; Arsphenamine, Chloramphenicol, Clindamycin, Lincomycin, Ethambutol, Fosfomycin, Fusidic acid, Furazolidone, Isoniazid, Linezolid, Metronidazole, Mupirocin, Nitrofurantoin, Platensimycin, Pyrazinamide, Quinupristin/Dalfopristin, Rifampicin (Rifampin in U.S.), Timidazole, Ropinerole, Ivermectin, Moxidectin, Afamelanotide, Cilengitide, or a combination thereof. In one aspect, the bioactive agent can be a combination of Rifampicin (Rifampin in U.S.) and Minocycline.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

Precipitated microparticles were prepared using an emulsion method. The microparticles were dried and subsequently treated with heptane to remove residual dichloromethane (DCM). The results are shown in Table 1. 1 h heptane treated microparticle refers to the duration that the microparticle was washed in heptane.

TABLE 1

| | | | Residual |
|---|---|---|---|
| | 24 h In Vitro | Residual | Heptane |
| Microparticle | Release, % | DCM, wt % | Content, wt % |
| Dried microparticle | 5.60 ± 0.13 | 3.23 | NA |
| | (RSD = 2.28%) | | |
| Post dry 1 h heptane treated | 2.50 ± 0.01 | 2.88 | 0.02 |
| microparticle | (RSD = 0.41%) | | |

### Example 2

Precipitated microparticles were prepared using an emulsion method. Without drying, the wet microparticles were treated with heptane to remove residual dichloromethane. The results are shown in Table 2. 0.5 h heptane treated microparticle refers to the duration that the microparticle was washed in heptane.

TABLE 2

| | | | Residual |
|---|---|---|---|
| | 24 h In Vitro | Residual | Heptane |
| Microparticle | Release, % | DCM, wt % | Content, wt % |
| Wet microparticle | 5.47 ± 0.04 | 2.78 | NA |
| | (RSD = 0.67%) | | |
| Post wet 0.5 h heptane | 3.32 ± 0.13 | 2.82 | None detected |
| treated microparticle | (RSD = 4.03%) | | |

### Example 3

Precipitated microparticles were prepared using an emulsion method. The microparticles were dried and subsequently treated with heptane to remove residual dichloromethane. The results are shown in Table 3. 2 h and 4 h heptane treated microparticles refers to the duration that the microparticle was washed in heptane.

TABLE 3

| | | | Residual |
|---|---|---|---|
| | 24 h In Vitro | Residual | Heptane |
| Microparticle | Release, % | DCM, wt % | Content, wt % |
| Dry microparticle | 2.06 ± 0.00 | 3.27 | NA |
| | (RSD = 0.24%) | | |
| Post dry 2 h heptane treated | 0.75 ± 0.04 | 2.86 | 0.12 |
| microparticle | (RSD = 4.84%) | | |
| Post dry 4 h heptane treated | 0.72 ± 0.02 | 2.89 | 0.11 |
| microparticle | (RSD = 2.92%) | | |

### Example 4

Precipitated microparticles were prepared using an emulsion method. The microparticles were dried and subsequently treated with heptane to remove residual dichloromethane. The results are shown in Table 1. 2 h heptane treated microparticle refers to the duration that the microparticle was washed in heptane.

TABLE 4

| | 24 h In Vitro | Residual | Residual |
|---|---|---|---|
| Microparticle | Release, % | DCM, wt % | Heptane, wt % |
| Dry microparticle | 46.59 ± 0.09 | 0.37 | <0.1 |
| | (RSD = 0.19%) | | |
| Post dry 2 h heptane | 63.97 ± 0.23 | <0.1 | 0.49 |
| treated microparticle | (RSD = 0.36%) | | |

### Example 5

Precipitated microparticles were prepared using an emulsion method. The microparticles were dried and subsequently treated with heptane to remove residual dichloromethane. The results are shown in Table 2. 2 h heptane treated microparticle refers to the duration that the microparticle was washed in heptane.

TABLE 5

| | 24 h In Vitro | Residual | Residual |
|---|---|---|---|
| Microparticle | Release, % | DCM, wt % | Heptane, wt % |
| Dry microparticle | 46.62 ± 0.63 | 1.15 | <0.1 |
| | (RSD = 1.35%) | | |
| Post dry 2 h heptane | 59.47 ± 0.44 | 0.39 | 0.11 |
| treated microparticle | (RSD = 0.74%) | | |

### Example 6

Precipitated microparticles were prepared using an emulsion method. The microparticles were dried and subsequently treated with heptane to remove residual dichloromethane. The results are shown in Table 3. 2 h heptane treated microparticle refers to the duration that the microparticle was washed in heptane.

TABLE 6

| | 24 h In Vitro | Residual | Residual |
|---|---|---|---|
| Microparticle | Release, % | DCM, wt % | Heptane, wt % |
| Dry microparticle | 21.57 ± 0.07 | 2.25 | <0.1 |
| | (RSD = 0.32%) | | |
| Post dry 2 h heptane | 13.53 ± 0.14 | 1.49 | <0.1 |
| treated microparticle | (RSD = 1.07%) | | |

Various modifications and variations can be made to the compounds, composites, kits, articles, devices, compositions, and methods described herein. Other aspects of the compounds, composites, kits, articles, devices, compositions, and methods described herein will be apparent from consideration of the specification and practice of the compounds, composites, kits, articles, devices, compositions, and methods disclosed herein. It is intended that the specification and examples be considered as exemplary.

What is claimed is:

1. A method for reducing residual organic solvent in microparticles, comprising:
(a) combining microparticles with a surfactant solution in a non-polar alkane to provide a dispersion;
(b) mixing the dispersion;
(c) collecting the microparticles;
(d) rinsing the microparticles; and
(e) drying the microparticles;
wherein prior to step (a), the microparticles comprise at least 2% of a residual organic solvent comprising a halogenated solvent, ethyl acetate, or a mixture thereof.

2. The method of claim 1, wherein the residual organic solvent comprises a C₁-C₄ halogenated alkane.

3. The method of claim 1, wherein the residual organic solvent comprises a C₁-C₄ chlorinated alkane.

4. The method of claim 1, wherein the residual organic solvent comprises methylene chloride, chloroform, carbon tetrachloride, ethylene dichloride, ethylene chloride, 2,2,2-trichloroethane, or a mixture thereof.

5. The method of claim 1, wherein the residual organic solvent comprises ethyl acetate.

6. The method of claim 1, wherein the surfactant comprises sorbitol monostearate, sorbitan monooleate, polyoxyethylene sorbitan monooleate, or a combination thereof.

7. The method of claim 1, wherein the microparticles are rinsed with a surfactant-free non-polar alkane, water, or a combination thereof.

8. The method of claim 1, wherein the microparticles are rinsed with surfactant-free heptane, water, or a combination thereof.

9. The method of claim 1, wherein the microparticles are collected by sieve separation.

10. The method of claim 1, wherein the microparticles comprise poly(lactide), poly(glycolide), poly(lactide-co-glycolide), or a copolymer, blend, or mixture thereof.

11. The method of claim 1, wherein the microparticles comprise a bioactive agent encapsulated therein.

12. The method of claim 1, wherein the bioactive agent is water-soluble.

13. The method of claim 1, wherein the bioactive agent comprises an oligopeptide.

14. The method of claim 1, wherein the bioactive agent comprises octreotide.

15. The method of claim 1, wherein the bioactive agent is at least partially soluble in the non-polar alkane.

16. The method of claim 1, wherein the bioactive agent has a solubility of at least 0.1 mg/mL in the non-polar alkane.
